(19) United States
(12) Patent Application Publication
(10) Pub. No.: US 2018/0142018 A1
(43) Pub. Date: May 24, 2018

(54) ANTI-CD19 ANTIBODIES AND METHODS OF USE THEREOF

(71) Applicant: NovImmune SA, Geneva (CH)

(72) Inventor: Nicolas Fischer, Geneva (CH)

(21) Appl. No.: 15/804,517

(22) Filed: Nov. 6, 2017

Related U.S. Application Data
(60) Provisional application No. 62/417,380, filed on Nov. 4, 2016.

(51) Int. Cl. C07K 16/28; A61K 47/68

(52) U.S. Cl. CPC C07K 16/2803; A61K 47/6849; C07K 2317/56; C07K 2317/565; C07K 2317/622; C07K 2317/33; C07K 2317/54; C07K 2317/569; C07K 2317/31; C07K 2317/24; C07K 2317/21; C07K 2317/55

(57) ABSTRACT

The disclosure relates to monoclonal antibodies and antigen binding fragments, variants, multimeric versions, or bispecifics thereof that specifically bind CD19, as well as methods of making and using these anti-CD19 antibodies and antigen binding fragments thereof in a variety of therapeutic, diagnostic and prophylactic indications.

### CROSS-REFERENCE TO RELATED APPLICATIONS

This application claims the benefit of U.S. Provisional Application No. 62/417,380, filed Nov. 4, 2016, the contents of which are incorporated herein by reference in its entirety.

### FIELD OF THE INVENTION

The disclosure relates to monoclonal antibodies and antigen binding fragments, variants, multimeric versions, or bispecifics thereof that specifically bind CD19, as well as methods of making and using these anti-CD19 antibodies and antigen binding fragments thereof in a variety of therapeutic, diagnostic and prophylactic indications.

### BACKGROUND OF THE INVENTION

B cells express a wide array of cell surface molecules during their differentiation and proliferation. Examples include the CD10, CD19, CD20, CD21, CD22, CD23, CD24, CD37, CD53, CD72, CD74, CD75, CD77, CD79a, CD79b, CD80, CD81, CD82, CD83, CD84, CD85, and CD86 leukocyte surface markers. These markers have been generally suggested as therapeutic targets for the treatment of B cell disorders or diseases, such as, for example, B cell malignancies, autoimmune diseases, and transplant rejection.

CD19 is a surface protein found on B cells and on certain cancerous cells derived from B cells, such as many B cell lymphomas. Anti-CD19 monoclonal antibodies have been generated in mice. However, mouse-derived antibodies are generally immunogenic in humans, and humanized antibodies may be immunogenic in humans.

Accordingly, there exists a need for fully human monoclonal antibodies and antigen-binding sequences thereof for use in therapeutics that target CD19.

### SUMMARY OF THE INVENTION

The disclosure provides monoclonal antibodies and antigen binding fragments or any fragments, variants, multimeric versions, or bispecifics thereof that bind CD19. These antibodies and antigen binding fragments or any fragments, variants, multimeric versions, or bispecifics thereof are collectively referred to herein as anti-CD19 monoclonal antibodies or anti-CD19 mAbs or antigen binding fragments or any fragments, variants, multimeric versions, or bispecifics thereof. Preferably, the monoclonal antibodies and antigen binding fragments or any fragments, variants, multimeric versions, or bispecifics thereof are specific for at least human CD19. In some embodiments, the monoclonal antibodies and antigen binding fragments or any fragments, variants, multimeric versions, or bispecifics thereof that recognize human CD19 are also cross-reactive for at least one other non-human CD19 protein, such as, by way of non-limiting example, non-human primate CD19, e.g., cynomolgus monkey CD19, and/or rodent CD19.

In some embodiments, the anti-CD19 monoclonal antibody or antigen binding fragments or any fragments, variants, multimeric versions, or bispecifics thereof includes a variable heavy chain amino acid sequence that is at least 90%, 91%, 92%, 93%, 94%, 95%, 96%, 97%, 98%, 99% or more identical to an amino acid sequence selected from SEQ ID NO: 2, 6, 12, 16, and 20. In some embodiments, the anti-CD19 monoclonal antibody or antigen binding fragments or any fragments, variants, multimeric versions, or bispecifics thereof includes a variable light chain amino acid sequence that is at least 90%, 91%, 92%, 93%, 94%, 95%, 96%, 97%, 98%, 99% or more identical to an amino acid sequence selected from SEQ ID NO: 4, 8, 10, 14, 18, and 22. In some embodiments, the anti-CD19 monoclonal antibody or antigen binding or any fragments, variants, multimeric versions, or bispecifics fragments thereof includes a variable heavy chain amino acid sequence that is at least 90%, 91%, 92%, 93%, 94%, 95%, 96%, 97%, 98%, 99% or more identical to an amino acid sequence selected from SEQ ID NO: 2, 6, 12, 16, and 20, and a variable light chain amino acid sequence that is at least 90%, 91%, 92%, 93%, 94%, 95%, 96%, 97%, 98%, 99% or more identical to an amino acid sequence selected from SEQ ID NO: 4, 8, 10, 14, 18, and 22.

In some embodiments, the anti-CD19 monoclonal antibody or antigen binding fragments or any fragments, variants, multimeric versions, or bispecifics thereof includes a variable heavy chain amino acid sequence comprising an amino acid sequence selected from SEQ ID NO: 2, 6, 12, 16, and 20. In some embodiments, the anti-CD19 monoclonal antibody or antigen binding or any fragments, variants, multimeric versions, or bispecifics fragments thereof includes a variable light chain amino acid sequence comprising an amino acid sequence selected from SEQ ID NO: 4, 8, 10, 14, 18, and 22. In some embodiments, the anti-CD19 monoclonal antibody or antigen binding or any fragments, variants, multimeric versions, or bispecifics fragments thereof includes a variable heavy chain amino acid sequence comprising an amino acid sequence selected from SEQ ID NO: 2, 6, 12, 16, and 20, and a variable light chain amino acid sequence comprising an amino acid sequence selected from SEQ ID NO: 4, 8, 10, 14, 18, and 22.

In some embodiments, the anti-CD19 monoclonal antibody or antigen binding fragments or any fragments, variants, multimeric versions, or bispecifics thereof includes a variable heavy chain complementarity determining region 1 (CDRH1) comprising the amino acid sequence of SEQ ID NO: 23 or 29, a variable heavy chain complementarity determining region 2 (CDRH2) comprising the amino acid sequence of SEQ ID NO: 24 or 30, and a variable heavy chain complementarity determining region 3 (CDRH3) comprising the amino acid sequence of SEQ ID NO: 25, 26, 27, 28, or 31.

In some embodiments, the anti-CD19 monoclonal antibody or antigen binding fragments or any fragments, variants, multimeric versions, or bispecifics thereof includes a variable light chain complementarity determining region 1 (CDRL1) comprising the amino acid sequence of SEQ ID NO: 32, 37, 41, or 44, a variable light chain complementarity determining region 2 (CDRL2) comprising the amino acid sequence selected of SEQ ID NO: 33, 38, 43, or 45, and a variable light chain complementarity determining region 3 (CDRL3) comprising the amino acid sequence of SEQ ID NO: 34, 35, 36, 40, 43, or 46.

In some embodiments, the anti-CD19 monoclonal antibody or antigen binding or any fragments, variants, multimeric versions, or bispecifics fragments thereof includes a variable heavy chain complementarity determining region 1 (CDRH1) comprising the amino acid sequence of SEQ ID NO: 23 or 29, a variable heavy chain complementarity determining region 2 (CDRH2) comprising the amino acid sequence of SEQ ID NO: 24 or 30, a variable heavy chain complementarity determining region 3 (CDRH3) comprising the amino acid sequence of SEQ ID NO: 25, 26, 27, 28, or 31, a variable light chain complementarity determining region 1 (CDRL1) comprising the amino acid sequence of SEQ ID NO: 32, 37, 41, or 44, a variable light chain complementarity determining region 2 (CDRL2) comprising the amino acid sequence selected of SEQ ID NO: 33, 38, 43, or 45, and a variable light chain complementarity determining region 3 (CDRL3) comprising the amino acid sequence of SEQ ID NO: 34, 35, 36, 40, 43, or 46.

The disclosure also provides monovalent antibodies or antigen binding fragments thereof that bind CD19. These antibodies or antigen binding fragments thereof are collectively referred to herein as anti-CD19 monovalent antibodies or anti-CD19 monov mAbs. The monovalent antibodies or antigen binding fragments thereof of the disclosure include one arm that specific recognizes CD19, and a second arm referred to herein as a dummy arm. The dummy arm includes an amino acid sequence that does not bind or otherwise cross-react with a human protein. In some embodiments, the dummy arm includes an amino acid sequence that does not bind or otherwise cross-react with a human protein that is found in whole blood. In some embodiments, the dummy arm includes an amino acid sequence that does not bind or otherwise cross-react with a human protein that is found in solid tissue. Preferably, the monovalent antibodies or antigen binding fragments thereof are specific for at least human CD19. In some embodiments, the monovalent antibodies or antigen binding fragments thereof that recognize human CD19 are also cross-reactive for at least one other non-human CD19 protein, such as, by way of non-limiting example, non-human primate CD19, e.g., cynomolgus monkey CD19, and/or rodent CD19. The anti-CD19 monovalent antibody or antigen binding fragments thereof can include any of the anti-CD19 binding sequences described herein. In some embodiments, the anti-CD19 monovalent antibody or antigen binding fragments thereof comprises an amino acid sequence that is from or is derived from an amino acid sequence in the 5F5 antibody, the 7F11 antibody, the 9G8 antibody, the F6 antibody, the 7F1 antibody, and the 10D8 antibody or any antigen binding fragment thereof described herein.

The antibodies of the disclosure that bind CD19 and fragments thereof serve to modulate, block, inhibit, reduce, antagonize, neutralize or otherwise interfere with the functional activity of CD19. Functional activities of CD19 include, by way of non-limiting example, functioning as a B cell co-receptor with CD21 and/or CD81, binding, when in the activated, phosphorylated state, to one or more Src-family kinases; and/or recruitment of PI-3 kinase. The antibodies are considered to completely modulate, block, inhibit, reduce, antagonize, neutralize or otherwise interfere with at least one functional activity of CD19 when the level of functional activity of CD19 in the presence of the antibody is decreased by at least 95%, e.g., by 96%, 97%, 98%, 99% or 100% as compared to the level of functional activity of CD19 in the absence of binding with an antibody described herein. The antibodies are considered to partially modulate, block, inhibit, reduce, antagonize, neutralize or otherwise interfere with at least one functional activity of CD19 when the level of functional activity of CD19 in the presence of the antibody is decreased by less than 95%, e.g., 10%, 20%, 25%, 30%, 40%, 50%, 60%, 75%, 80%, 85% or 90% as compared to the level of functional activity of CD19 in the absence of binding with an antibody described herein.

The disclosure also provides bispecific antibodies that recognize CD19 and a second target. In some embodiments, the second target is an antigen known to be associated or otherwise implicated in autoimmune diseases and/or inflammatory diseases, such as, for example, B-cell mediated autoimmune diseases and/or inflammatory diseases, including by way of non-limiting example, systemic lupus erythematosus (SLE), rheumatoid arthritis (RA), idiopathic thrombocytopenic purpura (ITP), Waldenstrom's hypergammaglobulinaemia, Sjogren's syndrome, multiple sclerosis (MS), and/or lupus nephritis.

The disclosure provides bispecific antibodies that recognize CD19 and a second target. The disclosure allows for the identification, production and purification of bispecific antibodies that are undistinguishable in sequence from standard antibodies and where one of the binding sites is specific for CD19 and the second binding site is specific for another target, for example a tumor-associated antigen (TAA). In some embodiments, the TAA is an antigen that is expressed on the cell surface of a cancer cell. In some embodiments, the cancer cell is selected from a lung cancer cell, a bronchial cancer cell, a prostate cancer cell, a breast cancer cell, a colorectal cancer cell, a pancreatic cancer cell, an ovarian, a leukemia cancer cell, a lymphoma cancer cell, an esophageal cancer cell, a liver cancer cell, a urinary and/or bladder cancer cell, a renal cancer cell, an oral cavity cancer cell, a pharyngeal cancer cell, a uterine cancer cell, and/or a melanoma cancer cell. In some embodiments, suitable second targets include, by way of non-limiting example, CD47, CD20, CD22, CD40, BAFFR, CD5, CD32b, ICOSL, IL6R, and/or IL21R.

The bispecific antibodies of the disclosure and antigen binding fragments thereof that recognize CD19 and a second target are generated using any methods known in the art such as, by way of non-limiting example, the κλ-body fully human bispecific antibody format described in PCT Publication No. WO 2012/023053, the use of cross-linked fragments, quadromas, and/or any of a variety of recombinant formats such as, by way of non-limiting examples, linked antibody fragments, forced heterodimers, and or recombinant formats based on single domains. Examples of Bispecific formats include but are not limited to bispecific IgG based on Fab arm exchange (Gramer et al., 2013 MAbs. 5(6)); the CrossMab format (Klein C et al., 2012 MAbs 4(6)); multiple formats based on forced heterodimerization approaches such as SEED technology (Davis J H et al., 2010 Protein Eng Des Sel. 23(4):195-202), electrostatic steering (Gunasekaran K et al., J Biol Chem. 2010 285(25):19637-46.) or knob-into-hole (Ridgway J B et al., Protein Eng. 1996 9(7):617-21.) or other sets of mutations preventing homodimer formation (Von Kreudenstein T S et al., 2013 MAbs. 5(5):646-54.); fragment based bispecific formats such as tandem scFv (such as BiTEs) (Wolf E et al., 2005 Drug Discov. Today 10(18):1237-44.); bispecific tetravalent antibodies (Pörtner L M et al., 2012 Cancer Immunol Immunother. 61(10):1869-75.); dual affinity retargeting molecules (Moore P A et al., 2011 Blood. 117(17):4542-51), diabodies (Kontermann R E et al., Nat Biotechnol. 1997 15(7):629-31).

The bispecific antibodies of the disclosure are generated using any methods known in the art such as, by way of non-limiting example, the use of cross-linked fragments, quadromas, and/or any of a variety of recombinant formats such as, by way of non-limiting examples, linked antibody fragments, forced heterodimers, and or recombinant formats based on single domains.

The monoclonal, monovalent and/or bispecific antibodies of the disclosure can be used for therapeutic intervention or as a research or diagnostic reagent. For example, the monoclonal, monovalent and/or bispecific antibodies of the disclosure are useful in methods of treating, preventing and/or delaying the progression of pathologies associated with aberrant CD19 expression and/or activity or alleviating a symptom associated with such pathologies, by administering an antibody of the disclosure to a subject in which such treatment or prevention is desired. The subject to be treated is, e.g., human. The monoclonal, monovalent and/or bispecific antibody is administered in an amount sufficient to treat, prevent, delay the progression or alleviate a symptom associated with the pathology.

In some embodiments, the monoclonal, monovalent and/or bispecific antibodies described herein are used in conjunction with one or more additional agents or a combination of additional agents. Suitable additional agents include current pharmaceutical and/or surgical therapies for an intended application, such as, for example, cancer, inflammation and/or autoimmune diseases. In some embodiments, the monoclonal, monovalent and/or bispecific antibodies can be used in conjunction with rituximab.

In some embodiments, the monoclonal, monovalent and/or bispecific antibodies and the additional agent are formulated into a single therapeutic composition, and the monoclonal, monovalent and/or bispecific antibody and additional agent are administered simultaneously. Alternatively, the ac monoclonal, monovalent and/or bispecific antibodies and additional agent are separate from each other, e.g., each is formulated into a separate therapeutic composition, and the monoclonal, monovalent and/or bispecific antibody and the additional agent are administered simultaneously, or the monoclonal, monovalent and/or bispecific antibodies and the additional agent are administered at different times during a treatment regimen. For example, the monoclonal, monovalent and/or bispecific antibody is administered prior to the administration of the additional agent, the monoclonal, monovalent and/or bispecific antibody is administered subsequent to the administration of the additional agent, or the monoclonal, monovalent and/or bispecific antibody and the additional agent are administered in an alternating fashion. As described herein, the monoclonal, monovalent and/or bispecific antibody and additional agent are administered in single doses or in multiple doses.

Pathologies treated and/or prevented using the antibodies of the disclosure include, for example, cancer or any other disease or disorder associated with aberrant CD19 expression and/or activity.

Pharmaceutical compositions according to the disclosure can include an antibody of the disclosure and a carrier. These pharmaceutical compositions can be included in kits, such as, for example, diagnostic kits.

### BRIEF DESCRIPTION OF THE DRAWINGS

FIGS. 1A-1F are a series of graphs depicting the ability of various anti-CD19 antibodies of the disclosure to bind six different B lymphocyte cell lines (Raji, Ramos, Nalm6, SU-DHL6, SU-DHL4, Mec2), a CD19-silenced cell line (Raji siRNA), and a negative control cell line (Jurkat), as determined by FACS analysis.

FIG. 2 is a series of graphs depicting the ability of various anti-CD19 antibodies of the disclosure to bind cynomolgus CD19 expressed by transfected CHO cells or a negative control cell line (CHO) as determined by FACS analysis.

FIGS. 3A and 3B are a series of graphs depicting the ability of various anti-CD19 antibodies of the disclosure at a concentration of 30 ug/mL or 3 ug/mL to bind human B lymphocytes.

FIGS. 3C and 3D are a series of graphs depicting the ability of various anti-CD19 antibodies of the disclosure at a concentration of 30 ug/mL or 3 ug/mL to bind cynomolgus B lymphocytes.

FIGS. 3E and 3F are a series of graphs depicting the ability of various anti-CD19 antibodies of the disclosure at a concentration of 30 ug/mL or 3 ug/mL to bind human T lymphocytes and monocytes.

### DETAILED DESCRIPTION

The disclosure provides monoclonal antibodies that bind CD19. These antibodies are collectively referred to herein as anti-CD19 monoclonal antibodies or anti-CD19 mAbs. Preferably, the monoclonal antibodies are specific for at least human CD19. In some embodiments, the monoclonal antibodies that recognize human CD19 are also cross-reactive for at least one other non-human CD19 protein, such as, by way of non-limiting example, non-human primate CD19, e.g., cynomolgus monkey CD19, and/or rodent CD19. The disclosure also includes antibodies that bind to the same epitope as an anti-CD19 monoclonal antibody disclosed herein.

The disclosure also provides monovalent antibodies and/or bispecific antibodies that include at least a first arm that is specific for CD19. Preferably, the monovalent antibodies and/or bispecific antibodies are specific for at least human CD19. In some embodiments, the monovalent antibodies and/or bispecific antibodies that recognize human CD19 are also cross-reactive for at least one other non-human CD19 protein, such as, by way of non-limiting example, non-human primate CD19, e.g., cynomolgus monkey CD19, and/or rodent CD19. The disclosure also provides antibodies that bind to the same epitope as an anti-CD19 monovalent and/or an anti-CD19 bispecific antibody disclosed herein.

The bispecific antibodies of the disclosure allow for simultaneous binding of the two antibody arms to two antigens on the surface of the cell (termed co-engagement), which results in additive or synergistic increase of affinity due to avidity mechanism. As a consequence, co-engagement confers high selectivity towards cells expressing both antigens as compared to cells that express just one single antigen. In addition, the affinities of the two arms of a bispecific antibody to their respective targets can be set up in a way that binding to target cells is principally driven by one of the antibody arms. In some embodiments, the bispecific antibody includes a first arm that binds CD19 and a second arm that binds a second target that is not CD19. In some embodiments, the bispecific antibody includes a first arm that binds CD19 and a second arm that binds a tumor associated antigen (TAA). In some embodiments, the bispecific antibody includes a first arm that binds CD19 and a second arm that binds a tumor associated antigen (TAA), where the first arm binds to CD19 with high affinity, and the second arm binds to the TAA with low affinity. In some embodiments, the TAA is an antigen that is expressed on the cell surface of a cancer cell. In some embodiments, the cancer cell is selected from a lung cancer cell, a bronchial cancer cell, a prostate cancer cell, a breast cancer cell, a colorectal cancer cell, a pancreatic cancer cell, an ovarian, a leukemia cancer cell, a lymphoma cancer cell, an esophageal cancer cell, a liver cancer cell, a urinary and/or bladder cancer cell, a renal cancer cell, an oral cavity cancer cell, a pharyngeal cancer cell, a uterine cancer cell, and/or a melanoma cancer cell. In some embodiments, suitable second targets include, by way of non-limiting example, CD47, CD20, CD22, CD40, BAFFR, CD5, CD32b, ICOSL, IL6R, and/or IL21R.

In some embodiments, the bispecific antibody is a fully human bispecific IgG format, such as the κλ-body format described in PCT Publication No. WO 2012/023053, the contents of which are incorporated by reference herein in their entirety.

Exemplary anti-CD19 monoclonal antibodies of the disclosure and antigen binding fragments thereof include, for example, the 5F5 antibody, the 7F11 antibody, the 9G8 antibody, the F6 antibody, the 7F1 antibody, and the 10D8 antibody or an antigen binding fragment thereof.

Exemplary anti-CD19 bispecific antibodies of the disclosure in which at least one binding site is specific for CD19 include, for example, the 5F5 antibody, the 7F11 antibody, the 9G8 antibody, the F6 antibody, the 7F1 antibody, and the 10D8 antibody or an antigen binding fragment thereof.

In some embodiments, exemplary anti-CD19 monoclonal antibodies of the disclosure and antigen binding fragments thereof include a combination of heavy chain complementarity determining regions (CDRs) selected from the CDR sequences shown in Table 1 and light chain CDRs selected from the CDR sequences shown in Table 2, where the CDRs shown in Tables 1 and 2 are defined according to the IMGT nomenclature.

In some embodiments, exemplary anti-CD19 monoclonal, monospecific anti-CD19 antibodies, anti-CD19 monovalent antibodies, and/or bispecific antibodies of the disclosure include a combination of heavy chain complementarity determining regions (CDRs) selected from the CDR sequences shown in Table 1 and light chain CDRs selected from the CDR sequences shown in Table 2, where the CDRs shown in Tables 1 and 2 are defined according to the IMGT nomenclature.

TABLE 1
Anti-CD19 Heavy Chain CDRs

| Antibody | CDRH1 | CDRH2 | CDRH3 |
|---|---|---|---|
| 5F5 | GYSFTSYW | IYPGDSDT | ARGISGIYNLHGFDI |
| | (SEQ ID | (SEQ ID NO: 24) | (SEQ ID NO: 25) |
| | NO: 23) | | |
| 7F11 | GYSFTSYW | IYPGDSDT | ARGVSGIYNLHGFDI |
| | (SEQ ID | (SEQ ID NO: 24) | (SEQ ID NO: 26) |
| | NO: 23) | | |
| 9G8 | GYSFTSYW | IYPGDSDT | ARGVSGIYNLHGFDI |
| | (SEQ ID | (SEQ ID NO: 24) | (SEQ ID NO: 26) |
| | NO: 23) | | |
| F6 | GYSFTSYW | IYPGDSDT | ARVWYYDFWSGADAFDI |
| | (SEQ ID | (SEQ ID NO: 24) | (SEQ ID NO: 27) |
| | NO: 23) | | |
| 7F1 | GYSFTSYW | IYPGDSDT | ARGDYWTGFAY |
| | (SEQ ID | (SEQ ID NO: 24) | (SEQ ID NO: 28) |
| | NO: 23) | | |
| 10D8 | GGTFSSYA | IIPIFGTA | ARDRGYDYVWGSYRY |
| | (SEQ ID | (SEQ ID NO: 30) | GAFDI |
| | NO: 29) | | (SEQ ID NO: 31) |

TABLE 2
Anti-CD19 Light Chain CDRs

| Antibody | CDRL1 | CDRL2 | CDRL3 |
|---|---|---|---|
| 5F5 | QSISSY | AAS | QQASLDSPLT |
| | (SEQ ID | (SEQ ID NO: 33) | (SEQ ID NO: 34) |
| | NO: 32) | | |
| 7F11 | QSISSY | AAS | QQGMWDNPFT |
| | (SEQ ID | (SEQ ID NO: 33) | (SEQ ID NO: 35) |
| | NO: 32) | | |
| 9G8 | QSISSY | AAS | QQGRFGSPFT |
| | (SEQ ID | (SEQ ID NO: 33) | (SEQ ID NO: 36) |
| | NO: 32) | | |
| F6 | QSVSSN | GAS | QQGSLEAPQT |
| | (SEQ ID | (SEQ ID NO: 38) | (SEQ ID NO: 40) |
| | NO: 37) | | |
| 7F1 | SSNIGNNY | DNN | GTWDLGWNSV |
| | (SEQ ID | (SEQ ID NO: 42) | (SEQ ID NO: 43) |
| | NO: 41) | | |
| 10D8 | SSDVGGYNY | EVS | SSYDVWVPHMV |
| | (SEQ ID | (SEQ ID NO: 45) | (SEQ ID NO: 46) |
| | NO: 44) | | |

### Anti-CD19 Antibodies

Exemplary anti-CD19 antibodies include the antibodies referred to herein as 5F5, 7F11, 9G8, F6, 7F1, and 10D8, or any fragments, variants, multimeric versions, or bispecifics thereof. Alternatively, the anti-CD19 antibody is an antibody or any fragments, variants, multimeric versions, or bispecifics thereof that binds to the same epitope as 5F5, 7F11, 9G8, F6, 7F1, and 10D8. These antibodies or any fragments, variants, multimeric versions, or bispecifics thereof are respectively referred to herein as “huCD19” antibodies. The huCD19 antibodies of the disclosure include fully human monoclonal antibodies, as well as humanized monoclonal antibodies and chimeric antibodies, or any fragments, variants, multimeric versions, or bispecifics thereof. These antibodies show specificity for human CD19, and they have been shown to modulate, e.g., block, inhibit, reduce, antagonize, neutralize or otherwise interfere with at least one biological function or activity of CD19.

Biological function or activities of CD19 include, by way of non-limiting example, functioning as a B cell co-receptor with CD21 and/or CD81, binding, when in the activated, phosphorylated state, to one or more Src-family kinases; and/or recruitment of PI-3 kinase. The antibodies are considered to completely modulate, block, inhibit, reduce, antagonize, neutralize or otherwise interfere with at least one functional activity of CD19 when the level of functional activity of CD19 in the presence of the antibody is decreased by at least 95%, e.g., by 96%, 97%, 98%, 99% or 100% as compared to the level of functional activity of CD19 in the absence of binding with an antibody described herein. The antibodies are considered to partially modulate, block, inhibit, reduce, antagonize, neutralize or otherwise interfere with at least one functional activity of CD19 when the level of functional activity of CD19 in the presence of the antibody is decreased by less than 95%, e.g., 10%, 20%, 25%, 30%, 40%, 50%, 60%, 75%, 80%, 85% or 90% as compared to the level of functional activity of CD19 in the absence of binding with an antibody described herein.

Each of the huCD19 monoclonal antibodies or any fragments, variants, multimeric versions, or bispecifics thereof described herein includes a heavy chain variable region (VH) and a light chain variable region (VL), as shown in the amino acid and corresponding nucleic acid sequences listed below. The CDR sequences, according to IMGT, are boxed in each of the VH and VL sequences below.

The 5F5 antibody includes a heavy chain variable region (VH) (SEQ ID NO: 2) encoded by the nucleic acid sequence shown in SEQ ID NO: 1, and a light chain variable region (VL) (SEQ ID NO: 4) encoded by the nucleic acid sequence shown in SEQ ID NO: 3:

>5F5_VH
(SEQ ID NO: 2)
EVQLVQSGAEVKKPGESLKISCKGSGYSFTSYWIGWVRQMPGKGLEWMGI
IYPGDSDTRYSPSFQGQVTISADKSISTAYLQWSSLKASDTAMYYCARGI
SGIYNLHGFDIWGQGTLVTVSS
>5F5_VH
(SEQ ID NO: 1)
GAGGTGCAGCTGGTGCAGTCTGGAGCAGAGGTGAAAAAGCCCGGGGAGTC
TCTGAAGATCTCCTGTAAGGGTTCTGGATACAGCTTTACCAGCTACTGGA
TCGGCTGGGTGCGCCAGATGCCCGGGAAAGGCCTGGAGTGGATGGGGATC
ATCTATCCTGGTGACTCTGATACCAGATACAGCCCGTCCTTCCAAGGCCA
GGTCACCATCTCAGCCGACAAGTCCATCAGCACCGCCTACCTTCAGTGGA
GCAGCCTGAAGGCCTCGGACACCGCCATGTATTACTGTGCGAGAGGTATA
AGTGGGATCTACAATTTACACGGTTTTGATATCTGGGGCCAGGGAACCCT
GGTCACAGTCTCGAGC
>5F5_VL
(SEQ ID NO: 4)
DIQMTQSPSSLSASVGDRVTITCRASQSISSYLNWYQQKPGKAPKLLIYA
ASSLQSGVPSRFSGSGSGTDFTLTISSLQPEDFATYYCQQASLDSPLTFG
QGTKVEIK
>5F5_VL
(SEQ ID NO: 3)
GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAGCAGGCGAGCTTGGACAGCCCGTTGACCTTCGGC
CAAGGGACCAAGGTGGAAATCAAA

The 7F11 antibody includes a heavy chain variable region (VH) (SEQ ID NO: 6) encoded by the nucleic acid sequence shown in SEQ ID NO: 5, and a light chain variable region (VL) (SEQ ID NO: 8) encoded by the nucleic acid sequence shown in SEQ ID NO: 7:

>7F11_VH
(SEQ ID NO: 6)
EVQLVQSGAEVKKPGESLKISCKGSGYSFTSYWIGWVRQMPGKGLEWMGI
IYPGDSDTRYSPSFQGQVTISADKSISTAYLQWSSLKASDTAMYYCARGV
SGIYNLHGFDIWGQGTLVTVSS
>7F11_VH
(SEQ ID NO: 5)
GAGGTGCAGCTGGTGCAGTCTGGAGCAGAGGTGAAAAAGCCCGGGGAGTC
TCTGAAGATCTCCTGTAAGGGTTCTGGATACAGCTTTACCAGCTACTGGA
TCGGCTGGGTGCGCCAGATGCCCGGGAAAGGCCTGGAGTGGATGGGGATC
ATCTATCCTGGTGACTCTGATACCAGATACAGCCCGTCCTTCCAAGGCCA
GGTCACCATCTCAGCCGACAAGTCCATCAGCACCGCCTACCTTCAGTGGA
GCAGCCTGAAGGCCTCGGACACCGCCATGTATTACTGTGCGAGAGGTGTA
AGTGGGATCTACAATTTACACGGTTTTGATATCTGGGGCCAGGGAACCCT
GGTCACAGTCTCGAGC
>7F11_VL
(SEQ ID NO: 8)
DIQMTQSPSSLSASVGDRVTITCRASQSISSYLNWYQQKPGKAPKLLIYA
ASSLQSGVPSRFSGSGSGTDFTLTISSLQPEDFATYYCQQGMWDNPFTFG
QGTKVEIK
>7F11_VL
(SEQ ID NO: 7)
GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAGCAGGGCATGTGGGACAACCCGTTCACCTTCGGC
CAAGGGACCAAGGTGGAAATCAAA

The 9G8 antibody includes a heavy chain variable region (VH) (SEQ ID NO: 6) encoded by the nucleic acid sequence shown in SEQ ID NO: 105, and a light chain variable region (VL) (SEQ ID NO: 10) encoded by the nucleic acid sequence shown in SEQ ID NO: 9:

>9G8_VH
(SEQ ID NO: 6)
EVQLVQSGAEVKKPGESLKISCKGSGYSFTSYWIGWVRQMPGKGLEWMGI
IYPGDSDTRYSPSFQGQVTISADKSISTAYLQWSSLKASDTAMYYCARGV
SGIYNLHGFDIWGQGTLVTVSS
>9G8_VH
(SEQ ID NO: 105)
GAGGTGCAGCTGGTGCAGTCTGGAGCAGAGGTGAAAAAGCCCGGGGAGTC
TCTGAAGATCTCCTGTAAGGGTTCTGGATACAGCTTTACCAGCTACTGGA
TCGGCTGGGTGCGCCAGATGCCCGGGAAAGGCCTGGAGTGGATGGGGATC
ATCTATCCTGGTGACTCTGATACCAGATACAGCCCGTCCTTCCAAGGCCA
GGTCACCATCTCAGCCGACAAGTCCATCAGCACCGCCTACCTTCAGTGGA
GCAGCCTGAAGGCCTCGGACACCGCCATGTATTACTGTGCGAGAGGTGTA
AGTGGGATCTACAATTTACACGGTTTCGATATCTGGGGCCAGGGAACCCT
GGTCACAGTCTCGAGC
>9G8_VL
(SEQ ID NO: 10)
DIQMTQSPSSLSASVGDRVTITCRASQSISSYLNWYQQKPGKAPKLLIYA
ASSLQSGVPSRFSGSGSGTDFTLTISSLQPEDFATYYCQQGRFGSPFTFG
QGTKVEIK
>9G8_VL
(SEQ ID NO: 9)
GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAGCAGGGCAGGTTCGGGTCCCCGTTCACCTTCGGC
CAAGGGACCAAGGTGGAAATCAAA

The F6 antibody includes a heavy chain variable region (VH) (SEQ ID NO: 12) encoded by the nucleic acid sequence shown in SEQ ID NO: 11, and a light chain variable region (VL) (SEQ ID NO: 14) encoded by the nucleic acid sequence shown in SEQ ID NO: 13:

>F6_VH
(SEQ ID NO: 12)
EVQLVQSGAEVKKPGESLKISCKGSGYSFTSYWIGWVRQMPGKGLEWMGI
IYPGDSDTRYSPSFQGQVTISADKSISTAYLQWSSLKASDTAMYYCARVW
YYDFWSGADAFDIWGQGTLVTVSS
>F6_VH
(SEQ ID NO: 11)
GAGGTGCAGCTGGTGCAGTCTGGAGCAGAGGTGAAAAAGCCCGGGGAGTC
TCTGAAGATCTCCTGTAAGGGTTCTGGATACAGCTTTACCAGCTACTGGA
TCGGCTGGGTGCGCCAGATGCCCGGGAAAGGCCTGGAGTGGATGGGGATC
ATCTATCCTGGTGACTCTGATACCAGATACAGCCCGTCCTTCCAAGGCCA
GGTCACCATCTCAGCCGACAAGTCCATCAGCACCGCCTACCTTCAGTGGA
GCAGCCTGAAGGCCTCGGACACCGCCATGTATTACTGTGCGAGAGTCTGG
TATTACGATTTTTGGAGTGGGGCCGATGCTTTTGATATCTGGGGCCAGGG
AACCCTGGTCACAGTCTCGAGC
>F6_VL
(SEQ ID NO: 14)
EIVMTQSPATLSVSPGERATLSCRASQSVSSNLAWYQQKPGQAPRLLIYG
ASTRATGIPARFSGSGSGTEFTLTISSLQSEDFAVYYCQQGSLEAPQTFG
QGTKVEIK
>F6_VL
(SEQ ID NO: 13)
GAAATAGTGATGACGCAGTCTCCAGCCACCCTGTCTGTGTCTCCAGGGGA
AAGAGCCACCCTCTCCTGCAGGGCCAGTCAGAGTGTTAGCAGCAACTTAG
CCTGGTACCAGCAGAAACCTGGCCAGGCTCCCAGGCTCCTCATCTATGGT
GCATCCACCAGGGCCACTGGTATCCCAGCCAGGTTCAGTGGCAGTGGGTC
TGGGACAGAGTTCACTCTCACCATCAGCAGCCTGCAGTCTGAAGATTTTG
CAGTTTATTACTGTCAGCAGGGCAGCTTGGAGGCGCCGCAGACCTTCGGC
CAAGGGACCAAGGTGGAAATCAAA

The 7F1 antibody includes a heavy chain variable region (VH) (SEQ ID NO: 16) encoded by the nucleic acid sequence shown in SEQ ID NO: 15, and a light chain variable region (VL) (SEQ ID NO: 18) encoded by the nucleic acid sequence shown in SEQ ID NO: 17:

>7F1_VH
(SEQ ID NO: 16)
EVQLVQSGAEVKKPGESLKISCKGSGYSFTSYWIGWVRQMPGKGLEWMGI
IYPGDSDTRYSPSFQGQVTISADKSISTAYLQWSSLKASDTAMYYCARGD
YWTGFAYWGQGTLVTVSS
>7F1_VH
(SEQ ID NO: 15)
GAGGTGCAGCTGGTGCAGTCTGGAGCAGAGGTGAAAAAGCCCGGGGAGTC
TCTGAAGATCTCCTGTAAGGGTTCTGGATACAGCTTTACCAGCTACTGGA
TCGGCTGGGTGCGCCAGATGCCCGGGAAAGGCCTGGAGTGGATGGGGATC
ATCTATCCTGGTGACTCTGATACCAGATACAGCCCGTCCTTCCAAGGCCA
GGTCACCATCTCAGCCGACAAGTCCATCAGCACCGCCTACCTGCAGTGGA
GCAGCCTGAAGGCCTCGGACACCGCCATGTATTACTGTGCGAGAGGTGAT
TATTGGACTGGTTTTGCTTATTGGGGCCAGGGAACCCTGGTCACAGTCTC
GAGC
>7F1_VL
(SEQ ID NO: 18)
QSVLTQPPSVSAAPGQKVTISCSGSSSNIGNNYVSWYQQLPGTAPKLLIY
DNNKRPSGIPDRFSGSKSGTSATLGITGLQTGDEADYYCGTWDLGWNSVF
GGGTKLTVL
>7F1_VL
(SEQ ID NO: 17)
CAGTCTGTGTTGACGCAGCCGCCCTCAGTGTCTGCGGCCCCAGGACAGAA
GGTCACCATCTCCTGCTCTGGAAGCAGCTCCAACATTGGGAATAATTATG
TATCCTGGTACCAGCAGCTCCCAGGAACAGCCCCCAAACTCCTCATTTAT
GACAATAATAAGCGACCCTCAGGGATTCCTGACCGATTCTCTGGCTCCAA
GTCTGGCACGTCAGCCACCCTGGGCATCACCGGACTCCAGACTGGGGACG
AGGCCGATTATTACTGCGGAACATGGGATCTGGGCTGGAACTCGGTGTTC
GGCGGAGGGACCAAGCTGACCGTCCTA

The 10D8 antibody includes a heavy chain variable region (VH) (SEQ ID NO: 20) encoded by the nucleic acid sequence shown in SEQ ID NO: 19, and a light chain variable region (VL) (SEQ ID NO: 22) encoded by the nucleic acid sequence shown in SEQ ID NO: 21:

>10D8_VH
(SEQ ID NO: 20)
QVQLVQSGAEVKKPGSSVKVSCKASGGTFSSYAISWVRQAPGQGLEWMGG
IIPIFGTANYAQKFQGRVTITADESTSTAYMELSSLRSEDTAVYYCARDR
GYDYVWGSYRYGAFDIWGQGTLVTVSS
>10D8_VH
(SEQ ID NO: 19)
CAGGTGCAGCTGGTGCAGTCTGGGGCTGAGGTGAAGAAGCCTGGGTCCTC
GGTGAAGGTCTCCTGCAAGGCTTCTGGAGGCACCTTCAGCAGCTATGCTA
TCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAG
AGTCACGATTACCGCGGACGAATCCACGAGCACAGCCTACATGGAGCTGA
GCAGCCTGAGATCTGAGGACACGGCCGTGTATTACTGTGCGAGAGATCGG
GGGTATGATTACGTTTGGGGGAGTTATCGTTATGGTGCCTTTGATATCTG
GGGCCAGGGAACCCTGGTCACAGTCTCGAGC
>10D8_VL
(SEQ ID NO: 22)
QSALTQPASVSGSPGQSITISCTGTSSDVGGYNYVSWYQQHPGKAPKLMI
YEVSNRPSGVSNRFSGSKSGNTASLTISGLQAEDEADYYCSSYDVWVPHM
VFGGGTKLTVL
>10D8_VL
(SEQ ID NO: 21)
CAGTCTGCCCTGACTCAGCCTGCCTCCGTGTCTGGGTCTCCTGGACAGTC
GATCACCATCTCCTGCACTGGAACCAGCAGTGACGTTGGTGGTTATAACT
ATGTCTCCTGGTACCAACAGCACCCAGGCAAAGCCCCCAAACTCATGATT
TATGAGGTCAGTAATCGGCCCTCAGGGGTTTCTAATCGCTTCTCTGGCTC
CAAGTCTGGCAACACGGCCTCCCTGACCATCTCTGGGCTCCAGGCTGAGG
ACGAGGCTGATTATTACTGCAGCTCATATGATGTCTGGGTCCCGCACATG
GTGTTCGGCGGAGGGACCAAGCTGACCGTCCTA

In some embodiments, the anti-CD19 antibody sequences presented herein or antigen binding fragments thereof are used to produce a monovalent antibody. The monovalent antibodies of the disclosure include a common heavy chain sequence, one arm that specifically recognizes CD19, and a second arm referred to herein as a dummy arm. The dummy arm includes an amino acid sequence that does not bind or otherwise cross-react with a human protein. In some embodiments, the dummy arm includes an amino acid sequence that does not bind or otherwise cross-react with a human protein that is found in whole blood. In some embodiments, the dummy arm includes an amino acid sequence that does not bind or otherwise cross-react with a human protein that is found in solid tissue. Preferably, the monovalent antibodies are specific for at least human CD19. In some embodiments, the monovalent antibodies that recognize human CD19 are also cross-reactive for at least one other non-human CD19 protein, such as, by way of non-limiting example, non-human primate CD19, e.g., cynomolgus monkey CD19, and/or rodent CD19.

In some embodiments, the anti-CD19 antibody sequence or an antigen binding fragment thereof is used with a second antibody sequence or an antigen binding fragment thereof that binds a target other than CD19 to produce a bispecific antibody referred to herein as an “anti-CD19 bispecific antibody.”

While antibody sequences below are provided herein as examples, it is to be understood that these sequences can be used to generate bispecific antibodies using any of a variety of art-recognized techniques. Examples of bispecific formats include but are not limited to fully human bispecific antibodies that include a common heavy chain, a kappa-type light chain, and a lambda-type light chain (PCT Publication No. WO 2012/023053), bispecific IgG based on Fab arm exchange (Gramer et al., 2013 MAbs. 5(6)); the CrossMab format (Klein C et al., 2012 MAbs 4(6)); multiple formats based on forced heterodimerization approaches such as SEED technology (Davis J H et al., 2010 Protein Eng Des Sel. 23(4):195-202), electrostatic steering (Gunasekaran K et al., J Biol Chem. 2010 285(25):19637-46.) or knob-into-hole (Ridgway J B et al., Protein Eng. 1996 9(7):617-21.) or other sets of mutations preventing homodimer formation (Von Kreudenstein T S et al., 2013 MAbs. 5(5):646-54.); fragment based bispecific formats such as tandem scFv (such as BiTEs) (Wolf E et al., 2005 Drug Discov. Today 10(18):1237-44.); bispecific tetravalent antibodies (Pörtner L M et al., 2012 Cancer Immunol Immunother. 61(10):1869-75.); dual affinity retargeting molecules (Moore P A et al., 2011 Blood. 117(17):4542-51), diabodies (Kontermann R E et al., Nat Biotechnol. 1997 15(7):629-31).

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, or any fragments, variants, multimeric versions, or bispecifics thereof, including, e.g., polyclonal, monoclonal, chimeric, dAb (domain antibody), single chain, F_{ab}, F_{ab′} and F_{(ab′)2} fragments, scFvs, and an F_{ab} expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody is the to specifically bind an antigen when the dissociation constant is ≤1 μM; e.g., ≤100 nM, preferably ≤10 nM and more preferably ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present disclosure is the to specifically bind to its target, when the equilibrium binding constant (K_{d}) is ≤1 μM, e.g., ≤100 nM, preferably ≤10 nM, and more preferably ≤1 nM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the disclosure include the nucleic acid molecules encoding the heavy chain immunoglobulin molecules, and nucleic acid molecules encoding the light chain immunoglobulin molecules described herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the disclosure comprise the heavy chain immunoglobulin molecules, and the light chain immunoglobulin molecules described herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

As used herein, the twenty conventional amino acids and their abbreviations follow conventional usage. See Immunology—A Synthesis (2nd Edition, E. S. Golub and D. R. Gren, Eds., Sinauer Associates, Sunderland Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyro sine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides ³H, ¹⁴C, ¹⁵N, ³⁵S, ⁹⁰Y, ⁹⁹Tc, ¹¹¹In, ¹²⁵I, ¹³¹I), (e.g., fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

### Antibodies

Various procedures known within the art may be used for the production of polyclonal or monoclonal antibodies directed against a given target, such as, for example, CD19, a tumor associated antigen or other target, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, N.Y., incorporated herein by reference).

Antibodies are purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

In some embodiments, the antibodies of the disclosure are monoclonal antibodies. Monoclonal antibodies are generated, for example, by using the procedures set forth in the Examples provided herein. Antibodies are also generated, e.g., by immunizing BALB/c mice with combinations of cell transfectants expressing high levels of a given target on their surface. Hybridomas resulting from myeloma/B cell fusions are then screened for reactivity to the selected target.

Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the disclosure can be readily isolated and sequenced using conventional procedures (e.g., by using oligonucleotide probes that are capable of binding specifically to genes encoding the heavy and light chains of murine antibodies). The hybridoma cells of the disclosure serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the disclosure, or can be substituted for the variable domains of one antigen-combining site of an antibody of the disclosure to create a chimeric bivalent antibody.

Monoclonal antibodies of the disclosure include humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization is performed, e.g., by following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies also comprise, e.g., residues which are found neither in the recipient antibody nor in the imported CDR or framework sequences. In general, the humanized antibody includes substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also includes at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Fully human antibodies are antibody molecules in which the entire sequence of both the light chain and the heavy chain, including the CDRs, arise from human genes. Such antibodies are termed “human antibodies”, or “fully human antibodies” herein. Monoclonal antibodies can be prepared by using trioma technique; the human B-cell hybridoma technique (see Kozbor, et al., 1983 Immunol Today 4: 72); and the EBV hybridoma technique to produce monoclonal antibodies (see Cole, et al., 1985 In: MONOCLONAL ANTIBODIES AND CANCER THERAPY, Alan R. Liss, Inc., pp. 77-96). Monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. An example of such a nonhuman animal is a mouse termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds specifically to the relevant epitope with high affinity are disclosed in PCT publication WO 99/53049.

The antibody can be expressed by a vector containing a DNA segment encoding the single chain antibody described above.

These can include vectors, liposomes, naked DNA, adjuvant-assisted DNA. gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired location. Additionally, the particles can be delivered by intracerebroventricular (icy) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

Bispecific antibodies are antibodies that have binding specificities for at least two different antigens. In the present case, one of the binding specificities is for a target such as CD19 or any fragment thereof. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Bispecific and/or monovalent antibodies of the disclosure can be made using any of a variety of art-recognized techniques, including those disclosed in co-pending application WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However it is also possible to obtain bispecific antibodies of the disclosure by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as Igκλ antibodies or “κλ bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

An essential step of the method is the identification of two antibody Fv regions (each composed by a variable light chain and variable heavy chain domain) having different antigen specificities that share the same heavy chain variable domain. Numerous methods have been described for the generation of monoclonal antibodies and fragments thereof. (See, e.g., Antibodies: A Laboratory Manual, Harlow E, and Lane D, 1988, Cold Spring Harbor Laboratory Press, Cold Spring Harbor, N.Y., incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies are generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of techniques are well-known in the art for producing xenogenic non-human animals. For example, see U.S. Pat. No. 6,075,181 and No. 6,150,584, which is hereby incorporated by reference in its entirety.

Alternatively, the antibodies are obtained by screening a library that contains antibody or antigen binding domain sequences for binding to the target antigen. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”).

Hybridomas resulting from myeloma/B cell fusions are then screened for reactivity to the target antigen. Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Although not strictly impossible, the serendipitous identification of different antibodies having the same heavy chain variable domain but directed against different antigens is highly unlikely. Indeed, in most cases the heavy chain contributes largely to the antigen binding surface and is also the most variable in sequence. In particular the CDR3 on the heavy chain is the most diverse CDR in sequence, length and structure. Thus, two antibodies specific for different antigens will almost invariably carry different heavy chain variable domains.

The methods disclosed in co-pending application WO 2012/023053 overcomes this limitation and greatly facilitates the isolation of antibodies having the same heavy chain variable domain by the use of antibody libraries in which the heavy chain variable domain is the same for all the library members and thus the diversity is confined to the light chain variable domain. Such libraries are described, for example, in co-pending applications WO 2010/135558 and WO 2011/084255, each of which is hereby incorporated by reference in its entirety. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the disclosure. The bispecific antibodies of the disclosure can be of different Isotypes and their Fc portion can be modified in order to alter the bind properties to different Fc receptors and in this way modify the effectors functions of the antibody as well as it pharmacokinetic properties. Numerous methods for the modification of the Fc portion have been described and are applicable to antibodies of the disclosure. (see for example Strohl, W R Curr Opin Biotechnol 2009 (6):685-91; U.S. Pat. No. 6,528,624; PCT/US2009/0191199 filed Jan. 9, 2009). The methods of the disclosure can also be used to generate bispecific antibodies and antibody mixtures in a F(ab′)2 format that lacks the Fc portion.

The common heavy chain and two different light chains are co-expressed into a single cell to allow for the assembly of a bispecific antibody of the disclosure. If all the polypeptides get expressed at the same level and get assembled equally well to form an immunoglobulin molecule then the ratio of monospecific (same light chains) and bispecific (two different light chains) should be 50%. However, it is likely that different light chains are expressed at different levels and/or do not assemble with the same efficiency. Therefore, a means to modulate the relative expression of the different polypeptides is used to compensate for their intrinsic expression characteristics or different propensities to assemble with the common heavy chain. This modulation can be achieved via promoter strength, the use of internal ribosome entry sites (IRES) featuring different efficiencies or other types of regulatory elements that can act at transcriptional or translational levels as well as acting on mRNA stability. Different promoters of different strength could include CMV (Immediate-early Cytomegalovirus virus promoter); EF1-1α (Human elongation factor 1α-subunit promoter); Ubc (Human ubiquitin C promoter); SV40 (Simian virus 40 promoter). Different IRES have also been described from mammalian and viral origin. (See e.g., Hellen C U and Sarnow P. Genes Dev 2001 15: 1593-612). These IRES can greatly differ in their length and ribosome recruiting efficiency. Furthermore, it is possible to further tune the activity by introducing multiple copies of an IRES (Stephen et al. 2000 Proc Natl Acad Sci USA 97: 1536-1541). The modulation of the expression can also be achieved by multiple sequential transfections of cells to increase the copy number of individual genes expressing one or the other light chain and thus modify their relative expressions. The Examples provided herein demonstrate that controlling the relative expression of the different chains is critical for maximizing the assembly and overall yield of the bispecific antibody.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the disclosure. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

Other suitable purification methods include those disclosed in co-pending application PCT/IB2012/003028, filed on Oct. 19, 2012, published as WO2013/088259, the contents of which are hereby incorporated by reference in their entirety.

In other embodiments of producing bispecific antibodies, antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface includes at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the disclosure. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate antibodies are also within the scope of the present disclosure. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the disclosure with respect to effector function, so as to enhance, e.g., the effectiveness of the antibody in treating cancer and/or other diseases and disorders associated with aberrant CD19 expression and/or activity. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

### Conjugated Antibodies

The disclosure also pertains to conjugated antibodies, also referred to herein as immunoconjugates, comprising an antibody or antigen-binding fragment thereof conjugated to a cytotoxic agent such as a toxin (e.g., an enzymatically active toxin of bacterial, fungal, plant, or animal origin, or fragments thereof), or a radioactive isotope (i.e., a radioconjugate).

In some embodiments, the toxin is a microtubule inhibitor or a derivative thereof. In some embodiments, the toxin is a dolastatin or a derivative thereof. In some embodiments, the toxin is auristatin E, AFP, MMAF, MMAE, MMAD, DMAF, or DMAE. In some embodiments, the toxin is a maytansinoid or maytansinoid derivative. In some embodiments, the toxin is DM1 or DM4. In some embodiments, the toxin is a nucleic acid damaging toxin. In some embodiments, the toxin is a duocarmycin or derivative thereof. In some embodiments, the toxin is a calicheamicin or a derivative thereof. In some embodiments, the agent is a pyrrolobenzodiazepine or a derivative thereof.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present disclosure, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Suitable linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present disclosure can be conjugated to the liposomes as described in Martin et al., J. Biol. Chem., 257: 286-288 (1982) via a disulfide-interchange reaction.

Use of Anti-CD19 Antibodies

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the disclosure, which include an antibody of the disclosure, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma The present disclosure also provides methods of treating or alleviating a symptom associated with a cancer. A therapeutic regimen is carried out by identifying a subject, e.g., a human patient suffering from (or at risk of developing) a cancer, using standard methods.

Therapeutic formulations of the disclosure, which include bispecific antibody of the disclosure that recognize CD19 and a second target are used to treat or alleviate a symptom associated with an autoimmune diseases and/or inflammatory diseases, such as, for example, B-cell mediated autoimmune diseases and/or inflammatory diseases, including by way of non-limiting example, systemic lupus erythematosus (SLE), rheumatoid arthritis (RA), idiopathic thrombocytopenic purpura (ITP), Waldenstrom's hypergammaglobulinaemia, Sjogren's syndrome, multiple sclerosis (MS), and/or lupus nephritis.

Efficaciousness of treatment is determined in association with any known method for diagnosing or treating the particular immune-related disorder. Alleviation of one or more symptoms of the immune-related disorder indicates that the antibody confers a clinical benefit.

Methods for the screening of antibodies that possess the desired specificity include, but are not limited to, enzyme linked immunosorbent assay (ELISA) and other immunologically mediated techniques known within the art.

Antibodies directed against a target such as CD19, a tumor associated antigen or other antigen (or a fragment thereof) may be used in methods known within the art relating to the localization and/or quantitation of these targets, e.g., for use in measuring levels of these targets within appropriate physiological samples, for use in diagnostic methods, for use in imaging the protein, and the like). In a given embodiment, antibodies specific any of these targets, or derivative, fragment, analog or homolog thereof, that contain the antibody derived antigen binding domain, are utilized as pharmacologically active compounds (referred to hereinafter as “Therapeutics”).

An antibody of the disclosure can be used to isolate a particular target using standard techniques, such as immunoaffinity, chromatography or immunoprecipitation. Antibodies of the disclosure (or a fragment thereof) can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the disclosure, including polyclonal, monoclonal, humanized and fully human antibodies, may be used as therapeutic agents. Such agents will generally be employed to treat or prevent a disease or pathology associated with aberrant expression or activation of a given target in a subject. An antibody preparation, preferably one having high specificity and high affinity for its target antigen, is administered to the subject and will generally have an effect due to its binding with the target. Administration of the antibody may abrogate or inhibit or interfere with the signaling function of the target. Administration of the antibody may abrogate or inhibit or interfere with the binding of the target with an endogenous ligand to which it naturally binds.

A therapeutically effective amount of an antibody of the disclosure relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the disclosure may be, by way of nonlimiting example, from about 0.1 mg/kg body weight to about 50 mg/kg body weight. Common dosing frequencies may range, for example, from twice daily to once a week.

Antibodies or a fragment thereof of the disclosure can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the disclosure can be used as an agent for detecting the presence of a given target (or a protein fragment thereof) in a sample. In some embodiments, the antibody contains a detectable label. Antibodies are polyclonal, or more preferably, monoclonal. An intact antibody, or a fragment thereof (e.g., F_{ab}, scFv, or F_{(ab)2}) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the disclosure can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Pharmaceutical Compositions

The antibodies of the disclosure (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The disclosure will be further described in the following examples, which do not limit the scope of the disclosure described in the claims.

### EXAMPLES

### Example 1: Lymphocyte Binding Analysis of Anti-CD19 Antibodies

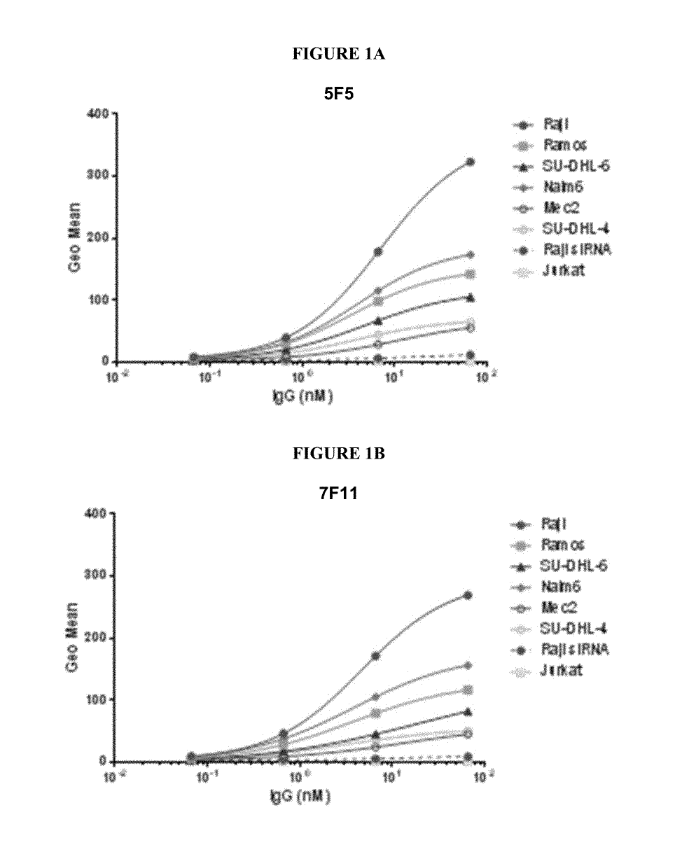

The ability of various anti-CD19 antibodies of the disclosure to bind various human B lymphocyte cell lines was evaluated. In particular, the human IgG1 5F5, 7F11, 9G8, F6, 7F1, and 10D8 anti-CD19 antibodies were evaluated for their abilities to bind (i) six human B lymphocyte cell lines: Raji, Ramos, Nalm6, Su-DHL6, Su-DHL4, and Mec2, (ii) a CD19 silenced B cell line: Raji siRNA; and a negative cell line (Jurkat). All incubations were prepared in FACS buffer (PBS, BSA 2%) at 4° C. Fc receptors on B cells were blocked with 10% mouse serum. Four doses of hIgG1 were tested: 10, 1, 0.1 and 0.01 μg/mL. Cell surface bound hIgG1 were detected with a mouse anti-human IgG Fc-PE mAb. The results of this study are shown in FIGS. 1A-1F.

As shown in FIGS. 1A-1F, all of the tested anti-CD19 antibodies bind to all of the six different B lymphocytes, although with different profiles and/or different affinities. For example, 7F1 and 10D8 bind better to Nalm6 than to Raji cells, whereas the other antibodies show the opposite binding profile. All the tested antibodies are clearly specific for CD19, and all of the tested antibodies lose the ability to bind CD19 in the CD19-silenced cell line Raji siRNA. None of the cell lines bound to the negative cell line.

### Example 2: Cross-Reactivity Analysis of Anti-CD19 Antibodies

The ability of the anti-CD19 antibodies to bind human and/or cynomolgus monkey CD19 was evaluated.

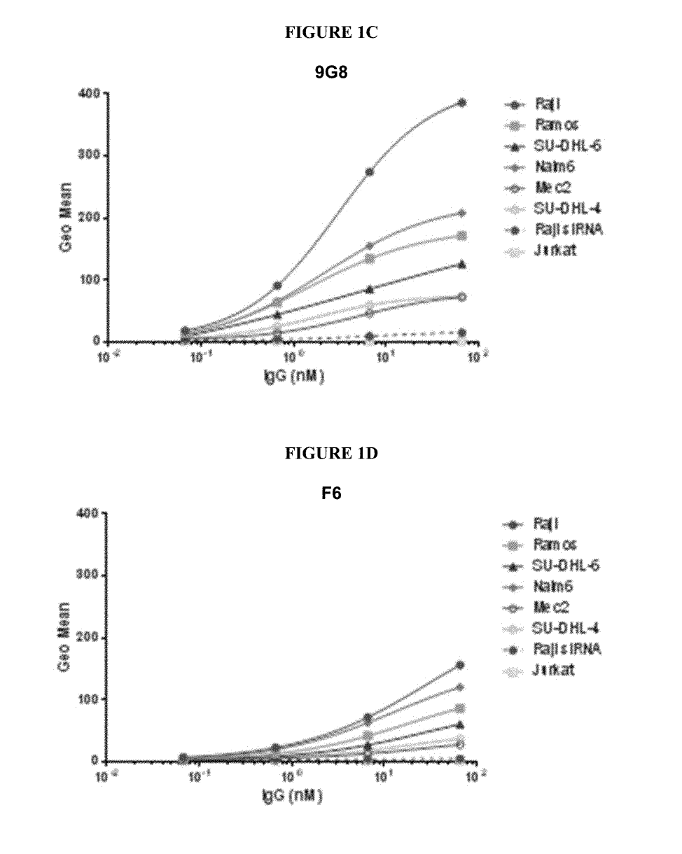

In particular, the human IgG1 5F5, 7F11, 9G8, F6, 7F1, and 10D8 anti-CD19 antibodies were evaluated for their abilities to bind a CHO cell line transfected with cynomolgus CD19 and a negative control cell line (CHO). All incubations were prepared in FACS buffer (PBS, BSA 2%) at 4° C. Fc receptors on B cells were blocked with 10% mouse serum. Four doses of hIgG1 were tested: 10, 1, 0.1 and 0.01 μg/mL. The results of this study are shown in FIG. 2.

The 9G8 anti-CD19 antibody is clearly cross-reactive with cynomolgus CD19. The anti-CD19 antibodies 5F5, 7F11, and F6 are also slightly cross-reactive with cynomolgus as seen on the FACS overlays in FIG. 2. The anti-CD19 antibodies 7F1 and 10D8 did not bind to the transfected CHO cynoCD19 cells.

### Example 3: Peripheral Blood Mononuclear Cell (PBMC) Binding Analysis of Anti-CD19 Antibodies

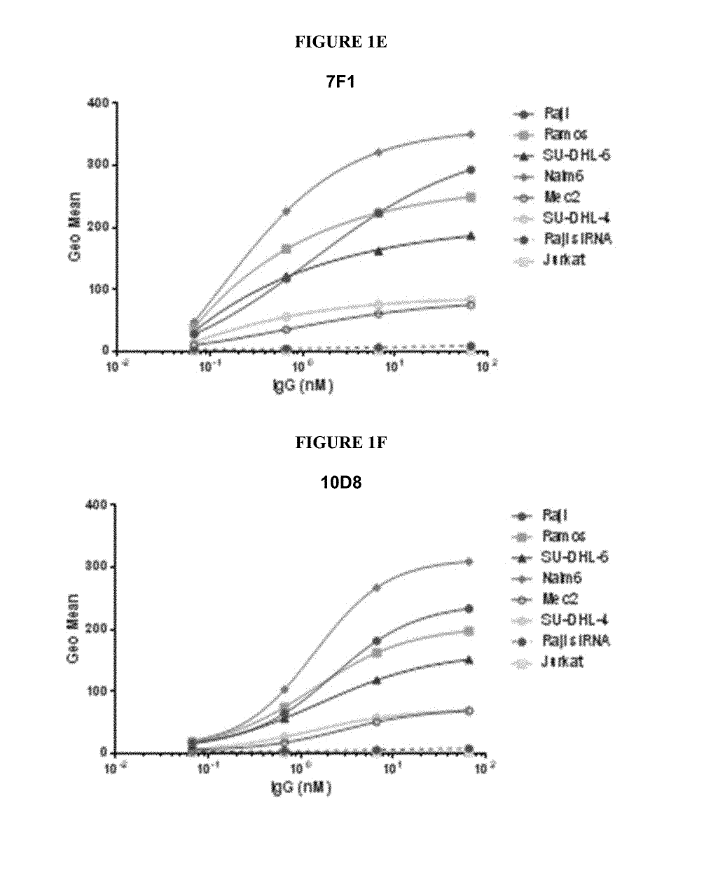

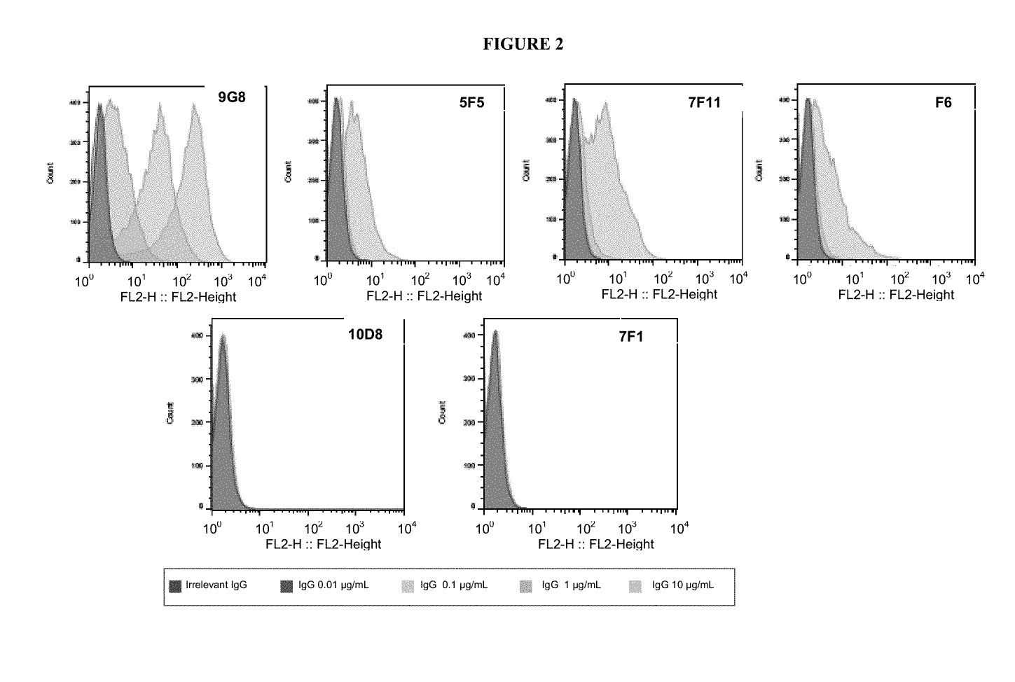

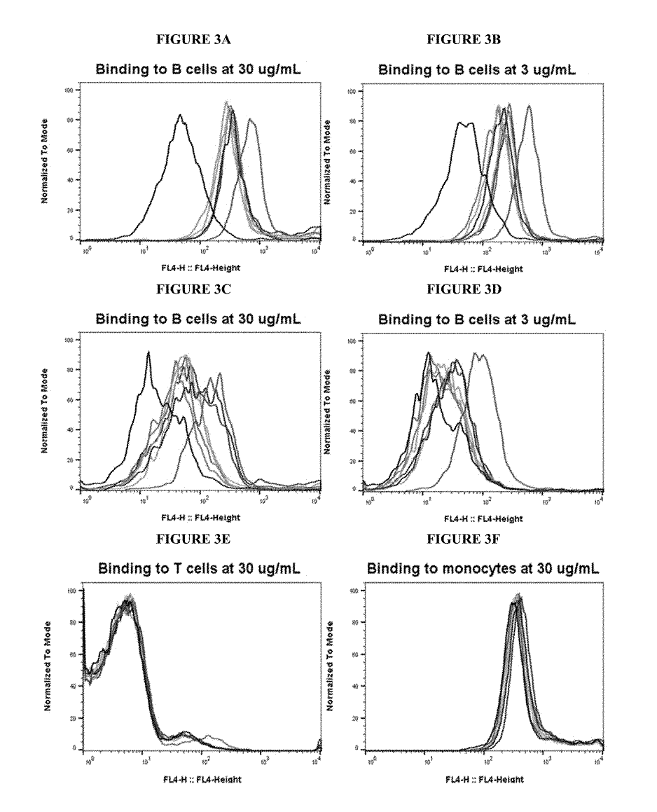

The ability of various anti-CD19 antibodies of the disclosure to bind various peripheral blood mononuclear cells (PBMC). Human and cynomolgus PBMC from frozen aliquots in citrate buffer were tested. Two doses of the following human IgG1 anti-CD19 antibodies were tested at 30 μg/mL and 3 μg/mL: 5F5, 7F11, 9G8, F6, 10D8, 7F1, Mdx as positive control, and an anti-IP-10 antibody referred to as NI-0801 as negative control. The PBMC were labelled with anti-CD20-PE monoclonal antibody (mAb), anti-CD14-FITC mAb, and anti-CD3-PerCP mAb cross-reactive both to human and cynomolgus species. Cell surface bound hIgG1 were detected with a mouse anti-human IgG Fc-APC mAb. FACS gating was performed with the anti-CD20 mAb for the B lymphocyte population, with the anti-CD3 for the T lymphocyte population and with anti-CD14 for the monocyte population. The results of these studies are shown in FIGS. 3A-3F.

As shown in FIG. 3E, none of the tested anti-CD19 antibodies bound to human PBMC CD3+ at a high dose of 30 μg/mL. All of the tested antibodies demonstrated the same binding level on monocytes as seen with the negative control NI-0801, binding due to cell surface Fc receptors. As shown in FIGS. 3A and 3B, all of the tested anti-CD19 antibodies bound to human PBMC CD20+, small affinity differences can be seen at 3 μg/mL. FIGS. 3C and 3D demonstrate the cross-reactivity of all the anti-CD19 antibodies. The binding is up to ten-fold higher on human than on cynomolgus PBMC, which is in the same range as seen for the positive control Mdx.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated monoclonal antibody or antigen binding fragment thereof that binds CD19 and comprises a combination of a variable heavy chain comprising a variable heavy chain complementarity determining region 1 (CDRH1) comprising the amino acid sequence of SEQ ID NO: 23 or 29, a variable heavy chain complementarity determining region 2 (CDRH2) comprising the amino acid sequence of SEQ ID NO: 24 or 30, a variable heavy chain complementarity determining region 3 (CDRH3) comprising the amino acid sequence of SEQ ID NO: 25, 26, 27, 28, or 31, a variable light chain complementarity determining region 1 (CDRL1) comprising the amino acid sequence of SEQ ID NO: 32, 37, 41, or 44, a variable light chain complementarity determining region 2 (CDRL2) comprising the amino acid sequence of SEQ ID NO: 33, 38, 43, or 45, and a variable light chain complementarity determining region 3 (CDRL3) comprising the amino acid sequence selected from SEQ ID NO: 34, 35, 36, 40, 43, or 46.

2. The isolated monoclonal antibody or antigen binding fragment thereof of claim 1 further comprising a combination of a variable heavy chain comprising the amino acid sequence of SEQ ID NO: 2, 6, 12, 16, or 20, and a variable light chain comprising the amino acid sequence of SEQ ID NO: 4, 8, 10, 14, 18, or 22.

3. The isolated monoclonal antibody or antigen binding fragment thereof of claim 1 further comprising a combination of a variable heavy chain sequence and a variable light chain sequence selected from the group consisting of:
(a) a variable heavy chain comprising the amino acid sequence of SEQ ID NO: 2 and a variable light chain comprising the amino acid sequence of SEQ ID NO: 4;
(b) a variable heavy chain comprising the amino acid sequence of SEQ ID NO: 6, and a variable light chain comprising the amino acid sequence of SEQ ID NO: 8;
(c) a variable heavy chain comprising the amino acid sequence of SEQ ID NO: 6 and a variable light chain comprising the amino acid sequence of SEQ ID NO: 10;
(d) a variable heavy chain comprising the amino acid sequence of SEQ ID NO: 12 and a variable light chain comprising the amino acid sequence of SEQ ID NO: 14;
(e) a variable heavy chain comprising the amino acid sequence of SEQ ID NO: 16 and a variable light chain comprising the amino acid sequence of SEQ ID NO: 18; and
(f) a variable heavy chain comprising the amino acid sequence of SEQ ID NO: 20 and a variable light chain comprising the amino acid sequence of SEQ ID NO: 22.

4. The isolated monoclonal antibody or antigen binding fragment thereof of claim 1, wherein the CD19 is human CD19.

5. The isolated monoclonal antibody or antigen binding fragment thereof of claim 1, wherein the antibody or antigen binding fragment thereof is a monoclonal antibody, a domain antibody (dAb), a single chain antibody (scAb), a Fab fragment, a F(ab′)₂ fragment, a single chain variable fragment (scFv), a scFv-Fc fragment, a single domain heavy chain antibody, a single domain light chain antibody, a variant antibody, a multimeric antibody, or a bispecific antibody.

6. The isolated monoclonal antibody or antigen binding fragment thereof of claim 1, wherein the antibody or antigen binding fragment thereof is a rabbit, mouse, chimeric, humanized or fully human monoclonal antibody.

7. The isolated monoclonal antibody or antigen binding fragment thereof of claim 1, wherein the antibody or antigen binding fragment thereof is an IgG isotype.

8. The isolated monoclonal antibody or antigen binding fragment thereof of claim 1, wherein the antibody or antigen binding fragment thereof is an IgG1 isotype.

9. An isolated monoclonal antibody or an antigen binding fragment thereof that competes for specific binding to human CD19 with the isolated antibody or antigen binding fragment thereof of claim 1.

10. The isolated monoclonal antibody or antigen binding fragment thereof of claim 1, wherein the antibody or antigen binding fragment thereof is conjugated to an agent.

11. The isolated monoclonal antibody or antigen binding fragment thereof of claim 10, wherein the agent is a toxin.

12. The isolated monoclonal antibody or antigen binding fragment thereof of claim 11, wherein the toxin is selected from the group consisting of a dolastatin or derivative thereof, an auristatin or derivative thereof, a maytansinoid or a derivative thereof, a duocarmycin or derivative thereof, a calicheamicin or derivative thereof, and a pyrrolobenzodiazepine or a derivative thereof.

13. The isolated monoclonal antibody or antigen binding fragment thereof of claim 11, wherein toxin is conjugated to the antibody or antigen binding fragment thereof via a linker.

14. The isolated monoclonal antibody or antigen binding fragment thereof of claim 13, wherein the linker is a cleavable linker.

15. An isolated bispecific antibody comprising a first arm that comprises a first amino acid sequence that binds CD19 and a second arm that comprises a second amino acid sequence that does not bind CD19.

16. The isolated bispecific antibody of claim 15, wherein the bispecific antibody comprises at least a first arm that binds CD19 and comprises a combination of a variable heavy chain comprising a variable heavy chain complementarity determining region 1 (CDRH1) comprising the amino acid sequence of SEQ ID NO: 23 or 29, a variable heavy chain complementarity determining region 2 (CDRH2) comprising the amino acid sequence of SEQ ID NO: 24 or 30, a variable heavy chain complementarity determining region 3 (CDRH3) comprising the amino acid sequence of SEQ ID NO: 25, 26, 27, 28, or 31, and a variable light chain comprising a variable light chain complementarity determining region 1 (CDRL1) comprising the amino acid sequence of SEQ ID NO: 32, 37, 41, or 44, a variable light chain complementarity determining region 2 (CDRL2) comprising the amino acid sequence of SEQ ID NO: 33, 38, 43, or 45, and a variable light chain complementarity determining region 3 (CDRL3) comprising the amino acid sequence selected from SEQ ID NO: 34, 35, 36, 40, 43, or 46.

17. The isolated bispecific antibody of claim 16, wherein the first arm that binds CD19 further comprises a combination of a variable heavy chain comprising the amino acid sequence of SEQ ID NO: 2, 6, 12, 16, or 20, and a variable light chain comprising the amino acid sequence of SEQ ID NO: 4, 8, 10, 14, 18, or 22.

18. The isolated bispecific antibody of claim 16, wherein the first arm that binds CD19 further comprises a combination of a variable heavy chain sequence and a variable light chain sequence selected from the group consisting of:
(a) a variable heavy chain comprising the amino acid sequence of SEQ ID NO: 2 and a variable light chain comprising the amino acid sequence of SEQ ID NO: 4;
(b) a variable heavy chain comprising the amino acid sequence of SEQ ID NO: 6, and a variable light chain comprising the amino acid sequence of SEQ ID NO: 8;
(c) a variable heavy chain comprising the amino acid sequence of SEQ ID NO: 6 and a variable light chain comprising the amino acid sequence of SEQ ID NO: 10;
(d) a variable heavy chain comprising the amino acid sequence of SEQ ID NO: 12 and a variable light chain comprising the amino acid sequence of SEQ ID NO: 14;
(e) a variable heavy chain comprising the amino acid sequence of SEQ ID NO: 16 and a variable light chain comprising the amino acid sequence of SEQ ID NO: 18; and
(f) a variable heavy chain comprising the amino acid sequence of SEQ ID NO: 20 and a variable light chain comprising the amino acid sequence of SEQ ID NO: 22.


SEQUENCE LISTING (105 sequences)

SEQ ID NO: 1 - DNA, 366 bp, Artificial Sequence
  Artificial Polynucleotide
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctaccttcagtggagcagcctgaaggcctcggacaccgccatgtattactgtgcgagaggtataagtgggatctacaatttacacggttttgatatctggggccagggaaccctggtcacagtctcgagc

SEQ ID NO: 2 - protein, 122 aa, Artificial Sequence
  Artificial Polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGISGIYNLHGFDIWGQGTLVTVSS

SEQ ID NO: 3 - DNA, 324 bp, Artificial Sequence
  Artificial Polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaggcgagcttggacagcccgttgaccttcggccaagggaccaaggtggaaatcaaa

SEQ ID NO: 4 - protein, 108 aa, Artificial Sequence
  Artificial Polypeptide
DIQMTQSPSSLSASVGDRVTITCRASQSISSYLNWYQQKPGKAPKLLIYAASSLQSGVPSRFSGSGSGTDFTLTISSLQPEDFATYYCQQASLDSPLTFGQGTKVEIK

SEQ ID NO: 5 - DNA, 366 bp, Artificial Sequence
  Artificial Polynucleotide
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctaccttcagtggagcagcctgaaggcctcggacaccgccatgtattactgtgcgagaggtgtaagtgggatctacaatttacacggttttgatatctggggccagggaaccctggtcacagtctcgagc

SEQ ID NO: 6 - protein, 122 aa, Artificial Sequence
  Artificial Polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGVSGIYNLHGFDIWGQGTLVTVSS

SEQ ID NO: 7 - DNA, 324 bp, Artificial Sequence
  Artificial Polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcagggcatgtgggacaacccgttcaccttcggccaagggaccaaggtggaaatcaaa

SEQ ID NO: 8 - protein, 108 aa, Artificial Sequence
  Artificial Polypeptide
DIQMTQSPSSLSASVGDRVTITCRASQSISSYLNWYQQKPGKAPKLLIYAASSLQSGVPSRFSGSGSGTDFTLTISSLQPEDFATYYCQQGMWDNPFTFGQGTKVEIK

SEQ ID NO: 9 - DNA, 324 bp, Artificial Sequence
  Artificial Polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcagggcaggttcgggtccccgttcaccttcggccaagggaccaaggtggaaatcaaa

SEQ ID NO: 10 - protein, 108 aa, Artificial Sequence
  Artificial Polypeptide
DIQMTQSPSSLSASVGDRVTITCRASQSISSYLNWYQQKPGKAPKLLIYAASSLQSGVPSRFSGSGSGTDFTLTISSLQPEDFATYYCQQGRFGSPFTFGQGTKVEIK

SEQ ID NO: 11 - DNA, 372 bp, Artificial Sequence
  Artificial Polynucleotide
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctaccttcagtggagcagcctgaaggcctcggacaccgccatgtattactgtgcgagagtctggtattacgatttttggagtggggccgatgcttttgatatctggggccagggaaccctggtcacagtctcgagc

SEQ ID NO: 12 - protein, 124 aa, Artificial Sequence
  Artificial Polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVWYYDFWSGADAFDIWGQGTLVTVSS

SEQ ID NO: 13 - DNA, 324 bp, Artificial Sequence
  Artificial Polynucleotide
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagggcagcttggaggcgccgcagaccttcggccaagggaccaaggtggaaatcaaa

SEQ ID NO: 14 - protein, 108 aa, Artificial Sequence
  Artifcial Polypeptide
EIVMTQSPATLSVSPGERATLSCRASQSVSSNLAWYQQKPGQAPRLLIYGASTRATGIPARFSGSGSGTEFTLTISSLQSEDFAVYYCQQGSLEAPQTFGQGTKVEIK

SEQ ID NO: 15 - DNA, 354 bp, Artificial Sequence
  Artificial Polynucleotide
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaggtgattattggactggttttgcttattggggccagggaaccctggtcacagtctcgagc

SEQ ID NO: 16 - protein, 118 aa, Artificial Sequence
  Artificial Polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGDYWTGFAYWGQGTLVTVSS

SEQ ID NO: 17 - DNA, 327 bp, Artificial Sequence
  Artificial Polynucleotide
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatctgggctggaactcggtgttcggcggagggaccaagctgaccgtccta

SEQ ID NO: 18 - protein, 109 aa, Artificial Sequence
  Artificial Polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLGWNSVFGGGTKLTVL

SEQ ID NO: 19 - DNA, 381 bp, Artificial Sequence
  Artificial Polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagatcgggggtatgattacgtttgggggagttatcgttatggtgcctttgatatctggggccagggaaccctggtcacagtctcgagc

SEQ ID NO: 20 - protein, 127 aa, Artificial Sequence
  Artificial Polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDRGYDYVWGSYRYGAFDIWGQGTLVTVSS

SEQ ID NO: 21 - DNA, 333 bp, Artificial Sequence
  Artificial Polynucleotide
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatgatgtctgggtcccgcacatggtgttcggcggagggaccaagctgaccgtccta

SEQ ID NO: 22 - protein, 111 aa, Artificial Sequence
  Artificial Polypeptide
QSALTQPASVSGSPGQSITISCTGTSSDVGGYNYVSWYQQHPGKAPKLMIYEVSNRPSGVSNRFSGSKSGNTASLTISGLQAEDEADYYCSSYDVWVPHMVFGGGTKLTVL

SEQ ID NO: 23 - protein, 8 aa, Artificial Sequence
  Artificial Polypeptide
GYSFTSYW

SEQ ID NO: 24 - protein, 8 aa, Artificial Sequence
  Artificial Polypeptide
IYPGDSDT

SEQ ID NO: 25 - protein, 15 aa, Artificial Sequence
  Artificial Polypeptide
ARGISGIYNLHGFDI

SEQ ID NO: 26 - protein, 15 aa, Artificial Sequence
  Artificial Polypeptide
ARGVSGIYNLHGFDI

SEQ ID NO: 27 - protein, 17 aa, Artificial Sequence
  Artificial Polypeptide
ARVWYYDFWSGADAFDI

SEQ ID NO: 28 - protein, 11 aa, Artificial Sequence
  Artificial Polypeptide
ARGDYWTGFAY

SEQ ID NO: 29 - protein, 8 aa, Artificial Sequence
  Artificial Polypeptide
GGTFSSYA

SEQ ID NO: 30 - protein, 8 aa, Artificial Sequence
  Artificial Polypeptide
IIPIFGTA

SEQ ID NO: 31 - protein, 20 aa, Artificial Sequence
  Artificial Polypeptide
ARDRGYDYVWGSYRYGAFDI

SEQ ID NO: 32 - protein, 6 aa, Artificial Sequence
  Artificial Polypeptide
QSISSY

SEQ ID NO: 33 - protein, 3 aa, Artificial Sequence
  Artificial Polypeptide
AAS

SEQ ID NO: 34 - protein, 10 aa, Artificial Sequence
  Artificial Polypeptide
QQASLDSPLT

SEQ ID NO: 35 - protein, 10 aa, Artificial Sequence
  Artificial Polypeptide
QQGMWDNPFT

SEQ ID NO: 36 - protein, 10 aa, Artificial Sequence
  Artificial Polypeptide
QQGRFGSPFT

SEQ ID NO: 37 - protein, 6 aa, Artificial Sequence
  Artificial Polypeptide
QSVSSN

SEQ ID NO: 38 - protein, 3 aa, Artificial Sequence
  Artificial Polypeptide
GAS

SEQ ID NO: 39 - skipped

SEQ ID NO: 40 - protein, 10 aa, Artificial Sequence
  Artificial Polypeptide
QQGSLEAPQT

SEQ ID NO: 41 - protein, 8 aa, Artificial Sequence
  Artificial Polypeptide
SSNIGNNY

SEQ ID NO: 42 - protein, 3 aa, Artificial Sequence
  Artificial Polypeptide
DNN

SEQ ID NO: 43 - protein, 10 aa, Artificial Sequence
  Artificial Polypeptide
GTWDLGWNSV

SEQ ID NO: 44 - protein, 9 aa, Artificial Sequence
  Artificial Polypeptide
SSDVGGYNY

SEQ ID NO: 45 - protein, 3 aa, Artificial Sequence
  Artificial Polypeptide
EVS

SEQ ID NO: 46 - protein, 11 aa, Artificial Sequence
  Artificial Polypeptide
SSYDVWVPHMV

SEQ ID NO: 47-104 - skipped (58 entries)

SEQ ID NO: 105 - DNA, 366 bp, Artificial Sequence
  Artificial Polynucleotide
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctaccttcagtggagcagcctgaaggcctcggacaccgccatgtattactgtgcgagaggtgtaagtgggatctacaatttacacggtttcgatatctggggccagggaaccctggtcacagtctcgagc